(19) United States
(12) Patent Application Publication
(10) Pub. No.: US 2025/0108170 A1
(43) Pub. Date: Apr. 3, 2025

(54) Sub-assembly for a medicament delivery device

(71) Applicant: SHL Medical AG, Zug (CH)

(72) Inventor: Anders Boström, Nacka Strand (SE)

(21) Appl. No.: 18/832,776

(22) PCT Filed: Jan. 16, 2023
(86) PCT No.: PCT/EP2023/050823

§ 371 Date: Jul. 24, 2024

(30) Foreign Application Priority Data
Jan. 28, 2022 (EP) 22154031.3

(51) Int. Cl. A61M 5/315

(52) U.S. Cl. CPC A61M 5/31511

(57) ABSTRACT

The present disclosure provides sub-assembly for a medicament delivery device, the sub-assembly comprising: a sub-assembly housing extending along a longitudinal axis between a distal end and a proximal end, wherein the sub-assembly housing comprises a flexible arm extending in a distal direction of the sub-assembly housing, and wherein the flexible arm comprises a distally directed flexible arm surface, a rotator comprising a support wall extending in the direction of the longitudinal axis, a plunger rod comprising a proximally directed plunger rod surface adjacent to the distally directed flexible arm surface, a biasing member extending in the direction of the longitudinal axis between a proximally directed sub-assembly housing surface of the sub-assembly housing and a distally directed surface of the plunger rod, and wherein the rotator is rotatable around the longitudinal axis relative to the sub-assembly housing from a first rotator position where the support wall is adjacent to an outer surface of the flexible arm of the sub-assembly housing to a second rotator position where the support wall is circumferentially offset to the outer surface of the flexible arm.

### CROSS-REFERENCE TO RELATED APPLICATIONS

The present application is a U.S. National Phase Application pursuant to 35 U.S.C. § 371 of International Application No. PCT/EP2023/050823 filed Jan. 16, 2023, which claims priority to European Patent Application No. 22154031.3 filed Jan. 28, 2022. The entire disclosure contents of these applications are herewith incorporated by reference into the present application.

### TECHNICAL FIELD

The present disclosure generally relates to medicament delivery devices.

### BACKGROUND

Medicament delivery devices such as autoinjectors may have a linearly movable needle cover extending from the housing. The needle is exposed when the needle cover is pushed into the housing. In some cases, the needle cover may also trigger medicament delivery.

There are medicament delivery devices that have a removable rear cap provided with a safety clip that extends radially into the housing and prevents the needle cover to move in the distal direction of the device, i.e., towards the rear end of the device.

When the rear cap with its safety clip has been removed, the needle cover can be moved further into the housing. When the needle cover is moved, it pushes a sleeve in the distal direction of the device. The plunger rod of the device, which prior to axial displacement of the sleeve is kept axially fixated by the sleeve, is thereby released.

### SUMMARY

An object of the present disclosure is to provide a sub-assembly for a medicament delivery device which solves, or at least mitigates, problems of the prior art.

There is according to a first aspect of the present disclosure provided a sub-assembly for a medicament delivery device, the sub-assembly comprising: a sub-assembly housing extending along a longitudinal axis between a distal end and a proximal end, wherein the sub-assembly housing comprises a flexible arm extending in a distal direction of the sub-assembly housing, and wherein the flexible arm comprises a distally directed flexible arm surface, a rotator comprising a support wall extending in the direction of the longitudinal axis, a plunger rod comprising a proximally directed plunger rod surface adjacent to the distally directed flexible arm surface of the flexible arm of the sub-assembly housing, a biasing member extending in the direction of the longitudinal axis between a proximally directed sub-assembly housing surface of the sub-assembly housing and a distally directed surface of the plunger rod, and wherein the rotator is rotatable around the longitudinal axis relative to the sub-assembly housing from a first rotator position where the support wall is adjacent to an outer surface of the flexible arm of the sub-assembly housing to a second rotator position where the support wall is circumferentially offset to the outer surface of the flexible arm.

The plunger rod is thus released from an axially fixed position by rotation of the rotator. An alternative solution to axial movement for releasing the plunger rod is thus provided. Further, the configuration may be more compact because it does not require axial displacement of the rotator.

The proximally directed plunger rod surface faces the distally directed flexible arm surface.

The proximally directed plunger rod surface may be configured to bear against the distally directed flexible arm surface when the rotator is in the first rotator position, to thereby prevent the plunger rod from axial movement in the proximal direction.

The flexible arm may be radially flexible.

The support wall may be arranged radially outside of the flexible arm when the rotator is in the first rotator to configured to prevent the flexible arm from flexing radially outwards.

The first arm may be allowed to flex radially outwards when the rotator is in the second rotator position.

The biasing member may be configured to bias the plunger rod in the proximal direction.

The first arm may be urged radially outwards by the plunger rod when the rotator is in the second rotator position, causing the distally directed flexible arm surface to move radially outwards so that it cease being in contact with the proximally directed plunger rod surface. The plunger rod from is thus released from engagement with the flexible arm.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment at least one of the distally directed flexible arm surface of the flexible arm and the proximally directed plunger rod surface of the plunger rod is bevelled.

According to one embodiment the flexible arm extends in the distal direction from the distal end of the sub-assembly housing.

According to one embodiment the rotator is axially immovable relative to the sub-assembly housing.

According to one embodiment the sub-assembly comprises an actuator member comprising an actuator surface, wherein the rotator comprises an actuated surface facing towards the actuator surface, wherein at least one of the actuator surface and the actuated surface is helical relative to the longitudinal axis, and wherein the actuator surface is configured to move along the actuated surface with a movement of the actuator member relative to the rotator in the direction of the longitudinal axis moving the rotator from the first rotator position to the second rotator position.

The rotator and the actuator member may thus form a cam mechanism, by which linear movement of the actuator member in the distal direction causes rotation of the rotator from the first rotator position to the second rotator position.

According to one embodiment the actuator surface is a distally directed surface and the actuated surface is a proximally directed surface.

According to one embodiment the actuated surface is helical between a proximal actuated surface end to a distal actuated surface end, and wherein the actuator member is axially movable relative to the rotator from a first axial position where the actuator surface is adjacent to the proximal actuated surface end to a second axial position where the actuator surface is adjacent to the distal actuated surface end.

According to one embodiment the rotator comprises a cylindrical or semi-cylindrical rotator body.

According to one embodiment the actuated surface of the rotator is arranged at the proximal end of the rotator body.

According to one embodiment the actuator member is a delivery member cover configured to cover a medicament delivery member.

According to one embodiment the plunger rod comprises a rod body, wherein a recess or cut-out is arranged in a sidewall of the rod body, and wherein a distal edge of the recess or cut-out defines the proximally directed surface.

According to one embodiment the biasing member is a compression spring surrounding a portion of the rod body of the plunger rod.

According to one embodiment the sub-assembly housing comprises an inner arm extending from the distal end towards the proximal end of the sub-assembly housing, wherein the inner arm comprises a protrusion radially inwardly extending from a proximal end of the inner arm, and wherein the plunger rod comprises a radially outwardly extending structure configured to interact with the protrusion of the inner arm when the plunger rod moves past the inner arm.

A mechanical click may thus be generated when medicament delivery is completed. This helps the user to understand that medicament delivery has been carried out.

According to one embodiment the radially outwardly extending structure extends from the rod body of the plunger rod, and wherein the radially outwardly extending structure has a surface that is perpendicular to the rod body.

There is according to a second aspect of the present disclosure provided a medicament delivery device comprising the sub-assembly of the first aspect, wherein the medicament delivery device is an autoinjector.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an example of a medicament delivery device comprising sub-assembly;

FIG. 2 is an exploded view of the medicament delivery device in FIG. 1;

FIG. 3 is a perspective view of a distal end of the medicament delivery device in FIG. 1 with its housing removed and a rear cap made transparent;

FIG. 4 is a side view of the medicament delivery device in FIG. 1 without its rear shell;

FIG. 5 is a close-up view of a distal end of the medicament delivery device shown in FIG. 4;

FIG. 6A is a side view of the medicament delivery device when the rotator is in a first rotator position;

FIG. 6B is a top view of the medicament delivery device in FIG. 6A;

FIG. 7A is a side view of the medicament delivery device when the rotator is in a second rotator position;

FIG. 7B is a top view of the medicament delivery device in FIG. 7A;

FIG. 8A shows a longitudinal section of a distal end portion of an example of a medicament delivery device before medicament delivery;

FIG. 8B shows a longitudinal section of a distal end portion of an example of a medicament delivery device after medicament delivery;

FIG. 9 is a perspective view of a variation of a medicament delivery device not forming part of the invention, without its rear shell, when the rotator is in the first rotational position;

FIG. 10 is a top view of the medicament delivery device shown in FIG. 9;

FIG. 11 is a close-up of a distal end of a plunger rod extending from a sub-assembly housing; and

FIG. 12 is top view of the medicament delivery device in FIG. 9 when the rotator is in the second rotational position.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

The sub-assembly disclosed herein is for a medicament delivery device. The medicament delivery device may for example be an autoinjector.

The sub-assembly comprises a sub-assembly housing having a proximal end and a distal end. The sub-assembly housing has at least one flexible arm.

The flexible arm is radially flexible.

The flexible arm extends in the distal direction from a distal end of a generally tubular body of the sub-assembly housing.

The sub-assembly comprises a rotator. The rotator is arranged concentrically with and around the sub-assembly housing.

The rotator is configured to be rotated about a longitudinal axis of the sub-assembly housing. The rotator is configured to be rotated from a first rotator position to a second rotator position.

The rotator comprises a support wall. The support wall extends in the direction of the longitudinal axis of the rotator.

The support wall is arranged radially outside of and adjacent to the flexible arm when the rotator is in the first rotator position.

The sub-assembly comprises a proximally biased plunger rod. The plunger rod has a proximally directed plunger rod surface bearing against a distally directed flexible arm surface of the flexible arm when the rotator is in the first rotator position. The plunger rod is thus prevented from moving in the proximal direction.

The support wall is arranged so close to the flexible arm when the rotator is in the first rotator position that it prevents the flexible arm from flexing radially outwards to a degree that would cause it to disengage from the plunger rod.

When the rotator is rotated from the first rotator position to the second rotator position, the support wall becomes circumferentially offset from the first arm. The first arm is thereby enabled to flex radially outwards and the proximally directed plunger rod surface ceases being in contact with the proximally directed surface of the flexible arm. The plunger rod is thus released to move in the proximal direction.

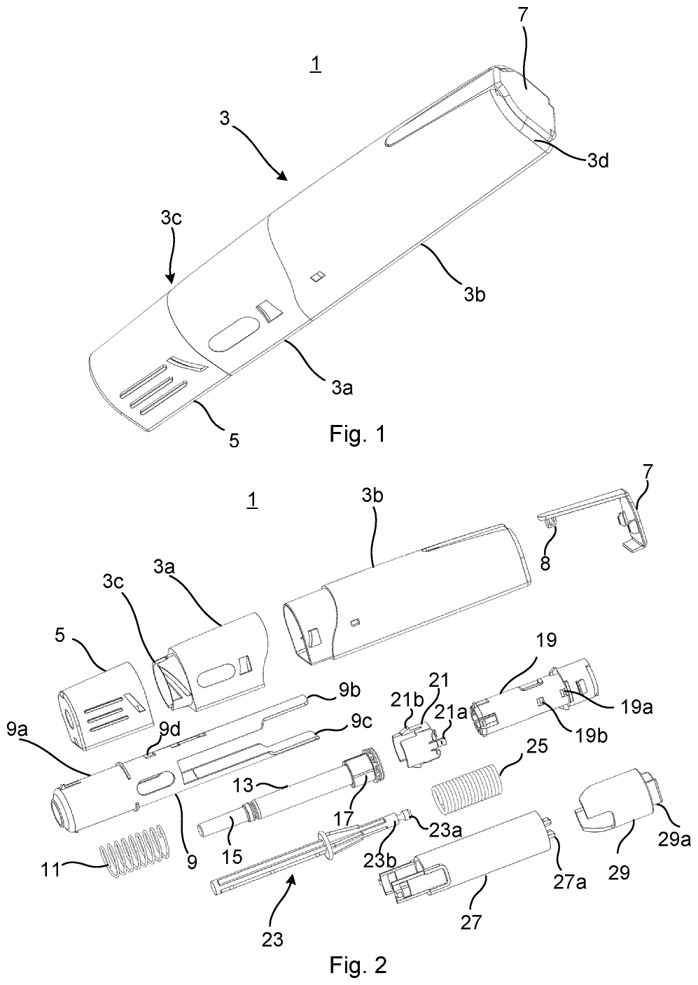

Examples of a medicament delivery device comprising the sub-assembly will now be described with reference to FIGS. 1-8B.

FIG. 1 shows a first example of a medicament delivery device 1. The medicament delivery device 1 is an autoinjector but could alternatively for example be a jet injector.

The medicament delivery device 1 comprises a housing 3.

The exemplified housing 3 comprises a front shell 3a and a rear shell 3b. The front shell 3a is connected to the rear shell 3b. The connection may for example be a snap fit connection. Alternatively, the housing 3 may consist of a single integral component.

The medicament delivery device 1 comprises a cap 5 configured to be detachably attached to the housing 3. In the example, the cap 5 is configured to be attached to the front shell 3a.

The housing 3 has a proximal housing end 3c, which in FIG. 1 is covered by the cap 5, and a distal housing end 3d.

The medicament delivery device 1 comprises a rear cap 7 removably attached to the housing 3. The rear cap 7 has a safety clip functionality, preventing activation of the medicament delivery device 1 when attached to the housing 3.

FIG. 2 is an exploded view of the medicament delivery device 1.

The medicament delivery device 1 comprises an actuator member 9. In the present example, the actuator member 9 is a delivery member cover, in the following designated by reference numeral 9. Alternatively, the actuator member 9 and the delivery member cover could be separate coaxially arranged components.

The delivery member cover 9 protrudes from the proximal housing end 3c.

The delivery member cover 9 is covered by the cap 5 when the cap 5 is attached to the housing 3. The delivery member cover 9 comprises a tubular proximal portion 9a and distally extending legs 9b and 9c extending from the tubular proximal portion 9a.

The delivery member cover 9 is movably arranged in the housing 3. The delivery member cover 9 is configured to be moved from an extended position to a retracted position relative to the housing 3. The delivery member cover 9 is received further by the housing 3 in the retracted position compared to the extended position.

The delivery member cover 9 can be moved from the extended position to the retracted position by pushing the medicament delivery device 1 against an injection site with the delivery member cover 9 directed towards the injection site.

The delivery member cover 9 is biased towards the extended position.

The medicament delivery device comprises a first resilient member 11 configured to bias the delivery member cover 9 towards the extended position. The first resilient member 11 may be a spring such as a compression spring.

The delivery member cover 9 is configured to return to the extended position from the retracted position when the force applied by the first resilient member 11 onto the delivery member cover 9 overcomes that applied onto the delivery member cover 9 by a user.

A medicament container 13 such as a syringe may be arranged in the medicament delivery device 1. The medicament container 13 may comprise a delivery member such as a needle.

Further, a delivery member shield 15 may be provided around the delivery member, e.g., the needle. The delivery member shield 15 may comprise a flexible needle shield and/or a rigid needle shield.

The exemplified medicament delivery device 1 comprises a medicament container support sleeve 17 arranged around a distal end portion of the medicament container 13. The medicament container support sleeve 17 may be arranged proximally of and adjacent to an end flange of the medicament container 13.

The medicament delivery device 1 comprises a medicament container holder 19. The medicament container holder 19 is axially fixed relative to the housing 3.

The medicament container support sleeve 17 is configured to be received by the medicament container holder 19 to maintain the medicament container 13 in an axially fixed relation with the medicament container holder 19. The medicament container holder 19 may be provided with an inner distally facing radial ledge configured to support a proximal end of the medicament container support sleeve 17.

The exemplified medicament delivery device 1 comprises a lock out collar 21. The lock out collar 21 is configured to be arranged radially outside the medicament container holder 19. The lock out collar 21 is configured to move axially relative to the medicament container holder 19, from an initial distal position when the delivery member cover 9 initially is in the extended position to a final proximal position when the delivery member cover 9 has returned to the extended position from the retracted position.

The lock out collar 21 has a grabbing arm 21a extending radially inwards. The grabbing arm 21a is configured to engage with a distal opening 19a of the medicament container holder 19 in the initial distal position when the delivery member cover 9 is initially in the extended position.

The lock out collar 21 has a radially outwards extending structure 21b arranged proximally axially offset from a radial opening 9d of the delivery member cover 9 when the delivery member cover 9 is in the extended position. The radial opening 9d is configured to receive the radially outwards extending structure 21b when the delivery member cover 9 is in the retracted position.

When the delivery member cover 9 is moved distally towards the retracted position, it moves relative to the lock out collar 21 to a position in which the radially outwards extending structure 21b radially aligns with and engages with the radial opening 9d. When the delivery member cover 9 is returned to the extended position, it brings with it the lock out collar 21. The grabbing arm 21a thus disengages from the distal opening 19a. When the delivery member cover 9 returns to the extended position the grabbing arm 21a engages with a proximal opening 19b of the medicament container holder 19, axially aligned with the distal opening 19a. The grabbing arm 21 has a distally facing radial surface and the proximal opening 19b has a proximally facing radial surface which faces the distally facing radial surface when the grabbing arm 21a has engaged with the proximal opening 19b. Thus, when the delivery member cover 9 has returned to the extended position, it is prevented from moving to the retracted position.

The medicament delivery device 1 comprises a plunger rod 23. The plunger rod 23 has a distal plunger rod end provided with a bud structure 23a.

The plunger rod 23 has a rod body and a recess or cut-out is arranged in a sidewall of the rod body. The bud structure 23a is formed by the recess or cut-out in the sidewall of the rod body. The plunger rod 23 extends into the medicament container 13.

The plunger rod 23 is biased in the proximal direction.

The medicament delivery device 1 comprises a biasing member 25, which is a second resilient member. The biasing member 25 may be a compression spring. The biasing member 25 is configured to bias the plunger rod 23 in the proximal direction.

The biasing member 25 surrounds a portion of the rod body of the plunger rod 23.

The medicament delivery device 1 comprises a sub-assembly housing 27. The sub-assembly housing 27 is configured to engage with the housing 3. The sub-assembly housing 27 is thus axially fixed relative to the housing 3. The sub-assembly housing 27 may for example be configured to engage with the housing 3 by means of a snap fit connection, or radial arms.

The biasing member 25 extends between a proximally directed sub-assembly housing surface, for example a radial surface forming an end wall of the generally cylindrical shape, of the sub-assembly housing 27, and a distally directed surface of the plunger rod 23.

The sub-assembly housing 27 has radially flexible arms 27a configured to engage with the plunger rod 23 to hold the plunger rod 23 in an initial axial position. The flexible arms 27a are configured to extend towards each other in a narrowed down region between the bud structure 23a and a distal shoulder 23b of the plunger rod 23, from which the bud structure 23a extends.

The flexible arms 27a extend in a distal direction from a distal end of the sub-assembly housing 27.

The medicament delivery device 1 comprises rotator 29. The rotator 29 is arranged concentrically with and radially outside the sub-assembly housing 27.

The rotator 29 is arranged axially immovable relative to the sub-assembly housing 27. For example, the rotator 29 may extend in the distal direction to an inner distal end surface of the housing 3 so that the rotator 29 is prevented to move in the distal direction.

The rotator 29 is rotatable from a first rotator position to a second rotator position.

The rotator 29 has a support wall 29a. The support wall 29a extends in the distal direction from a generally cylindrical or semi-cylindrical rotator body.

The support wall 29a is arranged radially outside an outer surface of one of the flexible arms 27a. The support wall 29a may be arc shaped.

The rotator 29 may comprise two support walls 29a facing each other. Each support wall 29a is in this case arranged radially outside an outer surface of a respective one of the flexible arms 27a. Each support wall 29a may be arc shaped.

The rotator 29 may be provided with inner radial recesses or radial openings between the two facing support walls 29a.

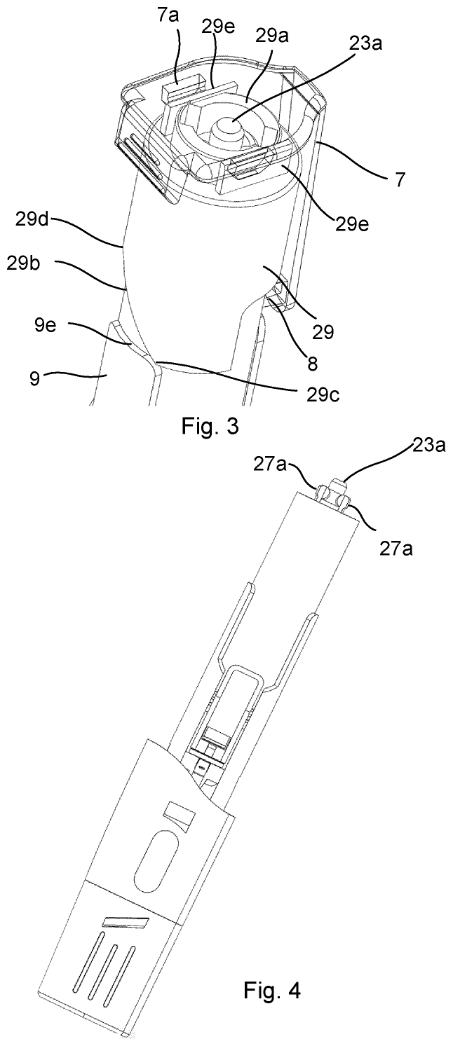

FIG. 3 shows the rear cap 7, made transparent, attached to the distal end of the medicament delivery device 1, with the housing 3 removed. The rear cap 7 has a protrusion 8, also shown in FIG. 2, extending radially inwards in between the delivery member cover 9 and the rotator 29 preventing axial movement of the delivery member cover 9 towards the retracted position as long as the rear cap 7 is attached to the housing 3.

The rotator 29 has an actuated surface 29b. The delivery member cover 9, or actuator member, has an actuator surface 9e. The actuated surface 29a faces the actuator surface 9e.

The actuated surface 29b is a proximally directed surface. The actuator surface 9e is a distally directed surface.

In the example, the actuated surface 29b is helical relative to the longitudinal axis of the sub-assembly. The actuated surface 29b is helical between a proximal actuated surface end 29c and a distal actuated surface end 29d.

Linear movement of the delivery member cover 9 from the extended position, in which the delivery member cover 9 is in a first axial position in which the actuator surface 9e is adjacent to the proximal actuated surface end 29c, towards the retracted position, causes rotation of the rotator 29 due to the cooperation between the actuated surface 29a and the actuator surface 9e. The delivery member cover 9 reaches a second axial position in the retracted position, in which the actuator surface 9e is adjacent to the distal actuated surface end 29d.

The rotator 29 and the rear cap 7 have a rotation prevention structure configured to prevent relative rotation between the rotator 29 and the rear cap 7. The rotator 29 may for example have axial structures 29e that extend in the distal direction from the generally cylindrical or semi-cylindrical rotator body radially outside the support walls 29a. The rear cap 7 may have corresponding axially extending structures 7a configured to be arranged radially outside of and adjacent to a respective axial structure 29e of the rotator 29 to prevent rotation of the rotator 29 as long as the rear cap 7 is attached to the housing 3. The delivery member cover 9 is thus also for this reason not able to move to the retracted position as long as the rear cap 7 is attached to the housing 3. The axial structures 29e may for example be planar structures, and the radially inwards extending structures 7a may extend radially to or essentially to the planar structures so that relative rotation between the rear cap 7 and the rotator 29 is restricted or prevented.

The distal housing end 3d may be provided with axial openings configured to receive a respective one of the structures 7a when the rear cap 7 is attached to the housing 3.

FIG. 4 shows the flexible arms 27a clamping the bud structure 23a, preventing the plunger rod 23 from movement in the proximal direction.

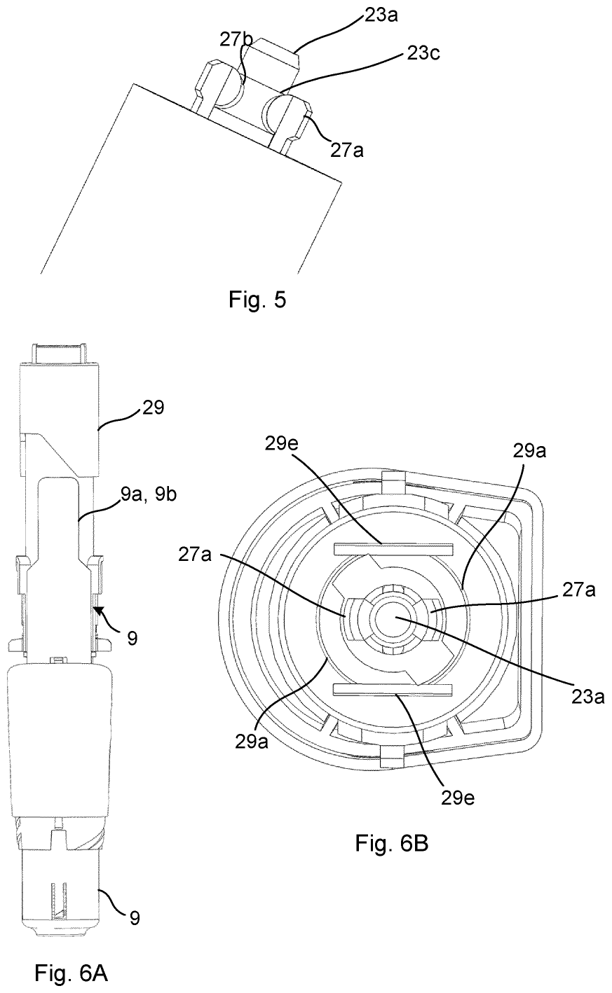

FIG. 5 is a close-up view of the flexible arms 27a and the bud structure 23a.

The plunger rod 23 has a proximally directed plunger rod surface 23c. Each flexible arm 27a has a distally directed flexible arm surface 27b which is arranged adjacent to and facing the proximally directed plunger rod surface 23c.

A distal edge of the cut-out or recess in the sidewall of the rod body defines the proximally directed plunger rod surface 23c.

The proximally directed plunger rod surface 23c may be bevelled or curved. The distally directed flexible arm surface 27b may be bevelled or curved.

FIG. 6A shows the medicament delivery device 1, exposing some internal components, when the delivery member cover 9 is in the extended position and the rotator 29 is in the first rotator position.

In the top/rear view of the medicament delivery device 1 shown in FIG. 6B, the support walls 29a are arranged radially outside of and adjacent to a respective one of the flexible arms 27a. The flexible arms 27a are thus prevented from flexing radially outwards and release their grip of the plunger rod 23.

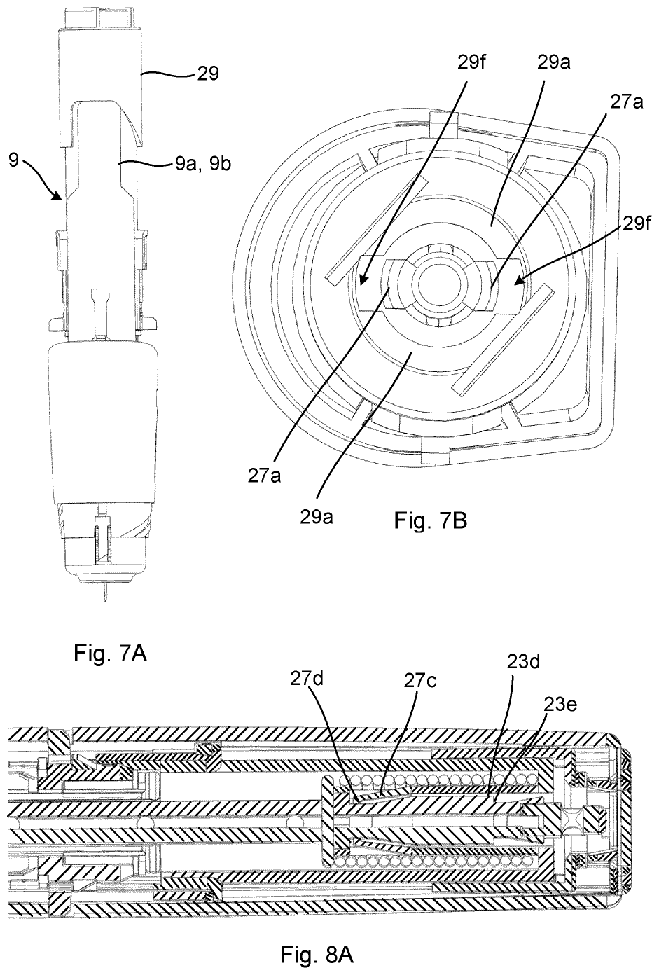

FIG. 7A shows the medicament delivery device 1 when the delivery member cover 9 is in the retracted position and the rotator 29 is in the second rotator position.

In the top/rear view of the medicament delivery device 1 shown in FIG. 7B, the support walls 29a are arranged circumferentially completely offset to a respective one of the flexible arms 27a. The flexible arms 27a are radially aligned with a respective one of the inner radial recesses or radial openings 29f between the two support walls 29a. The flexible arms 27a are thus able to flex radially outwards causing the release of the plunger rod 23 from the initial axial position.

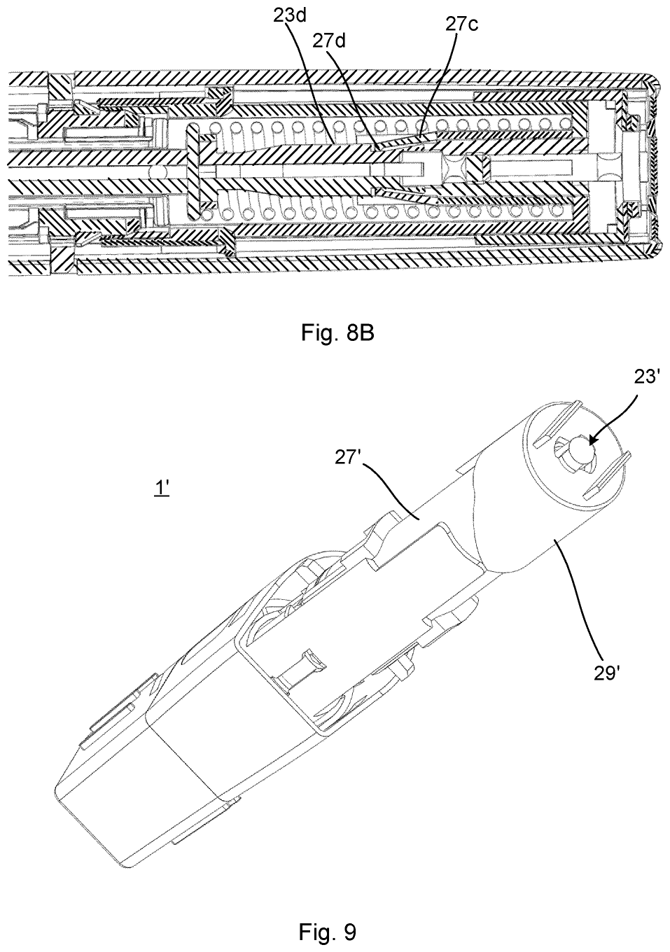

FIG. 8A shows a longitudinal section of a distal end portion of a variation of the medicament delivery device 1 provided with a mechanical click indication functionality indicating that medicament delivery has been completed, before medicament delivery.

The sub-assembly housing 27 comprises an inner arm 27c extending from the distal end towards the proximal end of the sub-assembly housing 27.

The inner arm 27c comprises a protrusion 27d radially inwardly extending from a proximal end of the inner arm 27c. The plunger rod 23 comprises a radially outwardly extending protrusion 23d configured to interact with the protrusion 27d of the inner arm 27c when the plunger rod 23 moves past the inner arm 27c, in particular when the radially outwardly extending protrusion 23d passes the protrusion 27d.

The radially outwardly extending structure 23d has an inclined surface that gradually increases the outer diameter of the plunger rod 23 in a direction towards the distal end of the plunger rod 23.

The radially outwardly extending structure 23d is initially arranged distally relative to the protrusion 27d, i.e., when the plunger rod 23 is in the initial axial position. When the plunger rod 23 is released from engagement with the flexible arms 27a, the plunger rod 23 is moved in the proximal direction, resulting in that the inner arm 27c is urged radially outwards due to contact with the outer surface of the radially outwardly extending structure 23d and its gradual increasing of the outer diameter of the plunger rod 23. The radially outwardly extending structure 23d has a distal end which is a radial step. The outer diameter of the plunger rod 23 is thus abruptly reduced. The radial step defines a surface 23e that is perpendicular to the outer surface of the rod body from which the radially outwardly extending protrusion 23d extends. After the radial step passes the protrusion 27d of the inner arm 27c, the inner arm 27c flexes back and the protrusion 27d impacts with the outer surface of the plunger rod 23. This causes an audible click which allows the user to understand that the medicament delivery has been completed.

FIGS. 9-12 show an example of a medicament delivery device 1′ comprising a sub-assembly similar to the sub-assembly described in conjunction with FIGS. 1-8B. The sub-assembly of the medicament delivery device 1′ is not part of the invention as defined by the claims.

The medicament delivery device 1′ comprises a sub-assembly that differs from that of the medicament delivery device 1.

The sub-assembly comprises a rotator 29′, a sub-assembly housing 27′, and a plunger rod 23′ that are slightly different from those previously described.

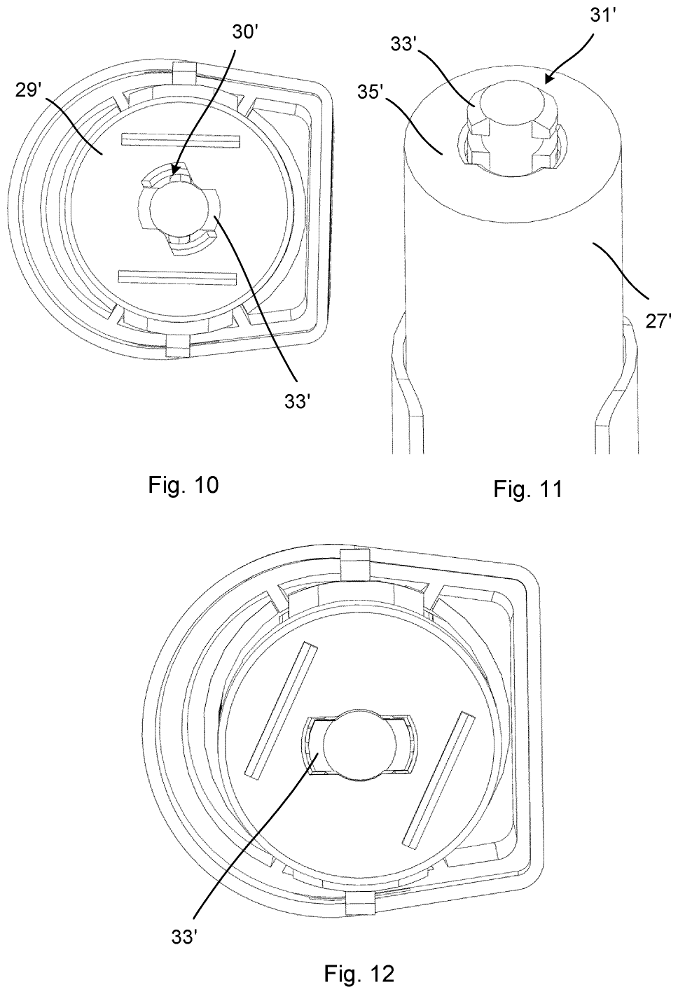

FIG. 10 shows a top/rear view of the medicament deliver device 1′ and FIG. 11 shows a distal end portion of the sub-assembly, with the rotator 29′ removed to illustrate the distal end of the plunger rod 23′.

The plunger rod 23′ has a distal end 31′ provided with radially outwardly extending protrusions 33′ which extend axially distally beyond a distal end surface 35′ of the sub-assembly housing 27′ before medicament delivery.

The distal end 31′ has a non-circular cross-sectional shape in cross-section through the longitudinal axis of the plunger rod 23′.

The distal end surface 35′ has a through-opening through which the distal end 31′ of the plunger rod 23′ extends. The through-opening has a non-circular cross-sectional shape in cross-section through the longitudinal axis of the sub-assembly housing 27′. The through-opening may have a cross-sectional shape corresponding to or matching the cross-sectional shape of the distal end 31′ of the plunger rod 23′ with its radially outwardly extending protrusions 33′ so that the distal end 31′ can pass through the through-opening.

The rotator 29′ has a rotator through-opening 30′ that is axially aligned with and of the same or similar cross-sectional shape and size as the through-opening of the sub-assembly housing 27′.

The distal end 31′ of the rotator 29′ extends through the rotator through-opening 30′ when the rotator 29′ is in the first rotator position.

The rotator 30′ is turned so relative to the plunger rod 23′ that the radially outwardly extending protrusions 33′ are circumferentially offset relative to the “keyhole” formed by the rotator through-opening 30′ when the rotator 29′ is in the first rotator position. The radially outwardly extending protrusions 33′ thus rest on a distal end surface of the rotator 29′.

FIG. 12 shows the medicament delivery device 1′ when the rotator 29′ is in the second rotator position. The rotator through-opening 30′ has become circumferentially aligned with the radially outwardly extending protrusions 33′ and the distal end 31′ can thus pass through the rotator through-opening 30′ and the through-opening of the sub-assembly housing 27′. The plunger rod 23′, which is proximally biased, is thus released and able to move in the proximal direction.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-15 (canceled)

16. A sub-assembly for a medicament delivery device, the sub-assembly comprising:
a sub-assembly housing extending along a longitudinal axis between a distal end and a proximal end, wherein the sub-assembly housing comprises a flexible arm extending in a distal direction of the sub-assembly housing, and wherein the flexible arm comprises a distally directed flexible arm surface;
a rotator comprising a support wall extending in the direction of the longitudinal axis;
a plunger rod comprising a proximally directed plunger rod surface adjacent to the distally directed flexible arm surface; and
a biasing member extending in the direction of the longitudinal axis between a proximally directed sub-assembly housing surface of the sub-assembly housing and a distally directed surface of the plunger rod,
wherein the rotator is rotatable around the longitudinal axis relative to the sub-assembly housing from a first rotator position where the support wall is adjacent to an outer surface of the flexible arm of the sub-assembly housing to a second rotator position where the support wall is circumferentially offset to the outer surface of the flexible arm.

17. The sub-assembly according to claim 16, wherein at least one of the distally directed flexible arm surface of the flexible arm and the proximally directed plunger rod surface of the plunger rod is bevelled.

18. The sub-assembly according to claim 16, wherein the flexible arm extends in the distal direction from the distal end of the sub-assembly housing.

19. The sub-assembly according to claim 16, wherein the rotator is axially immovable relative to the sub-assembly housing.

20. The sub-assembly according to claim 16, wherein the sub-assembly comprises an actuator member comprising an actuator surface, wherein the rotator comprises an actuated surface facing towards the actuator surface, wherein at least one of the actuator surface and the actuated surface is helical relative to the longitudinal axis, and wherein the actuator surface is configured to move along the actuated surface with a movement of the actuator member relative to the rotator in the direction of the longitudinal axis moving the rotator from the first rotator position to the second rotator position.

21. The sub-assembly according to claim 20, wherein the actuator surface is a distally directed surface and the actuated surface is a proximally directed surface.

22. The sub-assembly according to claim 21, wherein the actuated surface is helical between a proximal actuated surface end to a distal actuated surface end, and wherein the actuator member is axially movable relative to the rotator from a first axial position where the actuator surface is adjacent to the proximal actuated surface end to a second axial position where the actuator surface is adjacent to the distal actuated surface end.

23. The sub-assembly according to claim 16, wherein the rotator comprises a cylindrical or semi-cylindrical rotator body.

24. The sub-assembly according to claim 23, wherein the actuated surface of the rotator is arranged at the proximal end of the rotator body.

25. The sub-assembly according to claim 20, wherein actuator member is a delivery member cover configured to cover a medicament delivery member.

26. The sub-assembly according to claim 16, wherein the plunger rod comprises a rod body, wherein a recess or cut-out is arranged in a sidewall of the rod body, and wherein a distal edge of the recess or cut-out defines the proximally directed plunger rod surface.

27. The sub-assembly according to claim 26, wherein the biasing member is a compression spring surrounding a portion of the rod body of the plunger rod.

28. The sub-assembly according to claim 16, wherein the sub-assembly housing comprises an inner arm extending from the distal end towards the proximal end of the sub-assembly housing, wherein the inner arm comprises a protrusion radially inwardly extending from a proximal end of the inner arm, and wherein the plunger rod comprises a radially outwardly extending structure configured to interact with the protrusion of the inner arm when the plunger rod moves past the inner arm.

29. The sub-assembly according to claim 28, wherein the radially outwardly extending structure of the plunger rod extends from the rod body of the plunger rod, and wherein the radially outwardly extending structure has a surface that is perpendicular to the rod body.

30. A medicament delivery device comprising the sub-assembly according to claim 16, wherein the medicament delivery device is an autoinjector.
